(19) United States
(12) Patent Application Publication
(10) Pub. No.: US 2026/0241230 A1
(43) Pub. Date: Aug. 20, 2026

(54) LOW-LATENCY PERSONALIZED FITNESS RECOMMENDATION USING MUSCLE LOAD METHOD AND SYSTEM

(71) Applicant: Tata Consultancy Services Limited, Mumbai (IN)

(72) Inventors: SAJEELI GOYLE, Mumbai (IN); AVIK GHOSE, Kolkata (IN); OISHEE MAZUMDER, Kolkata (IN); MURALI PODUVAL, Thane (IN); NINAD YESHWANT RAJADHYAKSHA, Mumbai (IN); SWAPNA AGARWAL, Kolkata (IN)

(73) Assignee: Tata Consultancy Services Limited, Mumbai (IN)

(21) Appl. No.: 19/426,825

(22) Filed: Dec. 19, 2025

(30) Foreign Application Priority Data
Feb. 19, 2025 (IN) 202521014090

(51) Int. Cl. A63B 24/00; G16H 20/30

(52) U.S. Cl. CPC A63B 24/0075; G16H 20/30; A63B 2024/0068; A63B 2220/05; A63B 2220/54; A63B 2220/806; A63B 2230/60; A63B 2230/62

(57) ABSTRACT

This disclosure relates generally to low-latency personalized fitness recommendation using muscle load method and system. Recently, many technologies have been developed to monitor discrete aspects of individual state of health and fitness providing data metrics. In the new era of fitness, personalized workout plan provides incremental solution to achieve optimal health results. The method receives a video feed in real time and an user selected fitness activity along with user information to recommend personalized fitness workout. Further, from the video feed a current posture from body motion is detected to compute a muscle load. Furthermore, a target posture is predicted in reference to the user selected fitness activity. Finally, a personalized fitness workout is recommended by retrieving next exercise(s) for the target posture. Additionally, when no matching exercise(s) are identified in the exercise repository a GenAI fitness model synthetically generates a list of exercises.

### PRIORITY CLAIM

This U.S. patent application claims priority under 35 U.S.C. § 119 to: India application No. 202521014090, filed on Feb. 19, 2025. The entire contents of the aforementioned application are incorporated herein by reference.

### TECHNICAL FIELD

The disclosure herein generally relates to personalized fitness workout, and, more particularly, to low-latency personalized fitness recommendation using muscle load method and system.

### BACKGROUND

Achieving and maintaining physical fitness for all age groups helps maintain musculoskeletal health. increased physical activity is a key to better musculoskeletal health. In addition to increased physical activity, strengthening muscles and improving flexibility are also important for musculoskeletal health. Existing methods such as online fitness apps provides basic video search to retrieve exercise videos from a video library and the individual creates a workout plan based on their preferences with a bunch of exercises. However, such user goals promotes (muscle building, weight loss, weight gain etc.), preferences (home/gym, equipment, genre of exercise—yoga, Pilates etc.) and similar criteria but the method lack in assessing the way exercise was performed by the user. Also, personalized data inputs or user goals can only provide valuable insights to individual's physical needs and capabilities without validation of their performance.

In another existing method, video search is sometimes combined with a physical trainer, like a live streaming fitness session to provide some measure of personalized exercise recommendations. Exercises pre-stored in the video library may not be appropriate for the user but validations are limited. Such existing methods lack in providing fitness devices or systems integrated with artificial intelligence (AI) models to provide fully automated and comprehensive customized or personalized exercises workout plan constantly along with validating in real time each exercise performed by the use.

### SUMMARY

Embodiments of the present disclosure present technological improvements as solutions to one or more of the above-mentioned technical problems recognized by the inventors in conventional systems. For example, in one embodiment, a system for low-latency personalized fitness recommendation using muscle load is provided. The system includes receiving a video feed in real time and an user selected fitness activity along with user information, wherein the video feed comprises motion information of user per time instance captured via an image capturing device, and a set of exercises corresponding to the user selected fitness activity. Further, from the video feed, a current posture from body motion three dimensional (3D) coordinates of the user is dynamically detected starting from movelet and ending movelet, and joint-wise range of motion angles using a predefined muscle library. Then, a muscle load is computed for the current posture of the user selected fitness activity and an alert is raised if the muscle load exceeds a predefined muscle load for target muscle group.

Furthermore, a target posture for the user is predicted from the current posture in reference to the user selected fitness activity using (i) the muscle load, (ii) the user profile and an alert is raised for incorrect target posture performed by the user. The target posture is obtained from a exercise repository based on a matching criteria from pretrained biomechanical models, a correlation between the current posture of the user and the user selected fitness activity. Finally, a personalized fitness workout for the user is recommended by sequentially retrieving next best exercise(s) based on artificial intelligence (AI) recommendation, matching based on the target posture from the exercise repository, and wherein for absence of matching exercise(s) are identified in the exercise repository a generative artificial intelligence (GenAI) fitness model synthetically generates a list of exercises recommended by AI for the user.

In another aspect, a method for low-latency personalized fitness recommendation using muscle load is provided. The method includes receiving a video feed in real time and an user selected fitness activity along with user information, wherein the video feed comprises motion information of user per time instance captured via an image capturing device, and a set of exercises corresponding to the user selected fitness activity. Further, from the video feed a current posture from body motion three dimensional (3D) coordinates of the user is dynamically detected starting from movelet and ending movelet, and joint-wise range of motion angles are determined from computer vision, predefined models and stream of images from image capturing device. Then, a muscle load is computed for the current posture of the user selected fitness activity and an alert is raised if the muscle load exceeds a predefined muscle load for target muscle group.

Furthermore, a target posture for the user is predicted from the current posture in reference to the user selected fitness activity using (i) the muscle load, (ii) the user profile and an alert is raised for incorrect target posture performed by the user. The target posture is obtained from a exercise repository based on a matching criteria from pretrained biomechanical models, a correlation between the current posture of the user and the user selected fitness activity. Finally, a personalized fitness workout for the user is recommended by sequentially retrieving next best exercise(s) based on artificial intelligence (AI) recommendation, matching based on for the target posture from the exercise repository, and wherein for absence of matching exercise(s) are identified in the exercise repository a generative artificial intelligence (GenAI) fitness model synthetically generates a list of exercises recommended by AI for the user.

In yet another aspect, there are provided one or more non-transitory machine-readable information storage mediums comprising one or more instructions which when executed by one or more hardware processors cause: receiving a video feed in real time and an user selected fitness activity along with user information, wherein the video feed comprises motion information of user per time instance captured via an image capturing device, and a set of exercises corresponding to the user selected fitness activity. Further, from the video feed a current posture from body motion three dimensional (3D) coordinates of the user is dynamically detected starting from movelet and ending movelet, and joint-wise range of motion angles based on motion analysis of images from video capturing device. Then, a muscle load is computed for the current posture of the user selected fitness activity and an alert is raised if the muscle load exceeds a predefined muscle load for target muscle group.

Furthermore, a target posture for the user is predicted from the current posture in reference to the user selected fitness activity using (i) the muscle load, (ii) the user profile and an alert is raised for incorrect target posture performed by the user. The target posture is obtained from a exercise repository based on a matching criteria from pretrained biomechanical models, a correlation between the current posture of the user and the user selected fitness activity. Finally, a personalized fitness workout for the user is recommended by sequentially retrieving next best exercise(s) based on artificial intelligence (AI) recommendation, matching based on for the target posture from the exercise repository, and wherein for absence of matching exercise(s) are identified in the exercise repository a generative artificial intelligence (GenAI) fitness model synthetically generates a list of exercises recommended by AI for the user.

It is to be understood that both the foregoing general description and the following detailed description are exemplary and explanatory only and are not restrictive of the invention, as claimed.

### BRIEF DESCRIPTION OF THE DRAWINGS

The accompanying drawings, which are incorporated in and constitute a part of this disclosure, illustrate exemplary embodiments and, together with the description, serve to explain the disclosed principles:

FIG. 1 illustrates an exemplary system to recommend personalized fitness workout for a user according to some embodiments of the present invention.

FIG. 2 is a functional block diagram illustrating architecture overview of the system 100 for the personalized fitness workout, in accordance with some embodiments of the present disclosure.

FIG. 3 is a flow diagram illustrating a method to recommend personalized fitness workout using the system of FIG. 2, in accordance with some embodiments of the present disclosure.

FIG. 4 illustrates an use case example for personalized fitness workout recommendation by the system of FIG. 2, in accordance with some embodiments of the present disclosure.

### DETAILED DESCRIPTION OF EMBODIMENTS

Exemplary embodiments are described with reference to the accompanying drawings. In the figures, the left-most digit(s) of a reference number identifies the figure in which the reference number first appears. Wherever convenient, the same reference numbers are used throughout the drawings to refer to the same or like parts. While examples and features of disclosed principles are described herein, modifications, adaptations, and other implementations are possible without departing from the scope of the disclosed embodiments.

### Glossary

The term “breath analysis” refers to non-invasive diagnostic methods to detect inhaled gas-analytes.

“Ergonomics” refers to study of people in their working environment and various human factors.

“Personalized fitness workout” refers to tailored workout plans and guidance which helps to balance healthy lifestyle. It is designed based on users initial and specific goals.

“Muscle load” refers to the force exerted by an object on the muscle, and the force applied on the muscle refers to “muscle force”.

Embodiments herein provides a low-latency personalized fitness recommendation using muscle load method and system. The system 100 may be alternatively referred as personalized fitness recommender system. The system 100 is capable of providing an automated personalized fitness platform for user's personalized data input. Integrated specialized recommendation engine recommends a customized fitness workout(s). The method of the system 100 enables the user to provide real-time feedback for platform continual improvement. Initially, the user installs the automated personalized fitness application in any user device which communicates via a network with artificial cloud (AI) cloud. In response, the AI cloud recommends next best exercise(s) appropriate to achieve user's fitness goal. The method consumes the user profile (combination of flexibility and muscle strength) as input in addition to standard criteria of personalized fitness goal at scale. Furthermore, the method is not limited to extracts videos from a video library integrated with the AI cloud but synthetically generates relevant user goals using a skeletal body motion point coordinates and movements and biomechanics of the human body. Finally, the method extends to provide recommendation on premise exercises which also increases physical activity, flexibility and muscle strength. Additionally, the system 100 includes enhanced safety features for checking heart rate, ECG if available along with a widely used wearable devices.

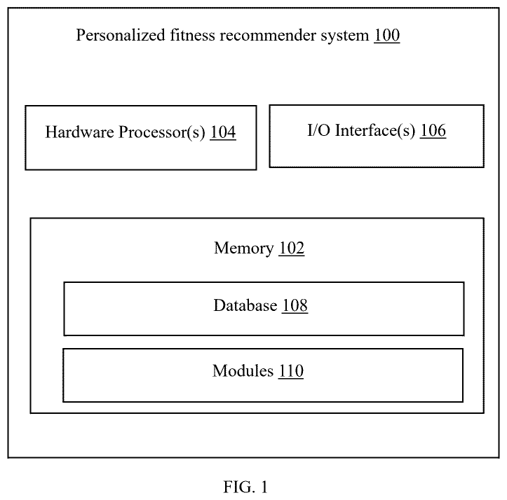

Referring now to the drawings, and more particularly to FIG. 1 through FIG. 4, where similar reference characters denote corresponding features consistently throughout the figures, there are shown preferred embodiments and these embodiments are described in the context of the following exemplary system and/or method.

FIG. 1 illustrates an exemplary system to recommend personalized fitness workout for a user according to some embodiments of the present invention. In an embodiment, the system 100 includes a processor(s) 104, communication interface device(s), alternatively referred as input/output (I/O) interface(s) 106, and one or more data storage devices or a memory 102 operatively coupled to the processor(s) 104. The system 100 with one or more hardware processors is configured to execute functions of one or more functional blocks of the system 100.

Referring to the components of system 100, in an embodiment, the processor(s) 104, can be one or more hardware processors 104. In an embodiment, the one or more hardware processors 104 can be implemented as one or more microprocessors, microcomputers, microcontrollers, digital signal processors, central processing units, state machines, logic circuitries, and/or any devices that manipulate signals based on operational instructions. Among other capabilities, the one or more hardware processors 104 are configured to fetch and execute computer-readable instructions stored in the memory 102. In an embodiment, the system 100 can be implemented in a variety of computing systems including laptop computers, notebooks, hand-held devices such as mobile phones, workstations, mainframe computers, servers, and the like.

The I/O interface(s) 106 may include a variety of software and hardware interfaces, for example, a user interface, a tracking dashboard to display performance of the enterprise application, and the like and can facilitate multiple communications within a wide variety of networks N/W and protocol types, including wired networks, for example, LAN, cable, etc., and wireless networks, such as WLAN, cellular and the like. In an embodiment, the I/O interface(s) 106 can include one or more ports for connecting to a number of external devices or to another server or devices. The system 100 receives inputs from user via an application installed on user-end devices connected to the system 100 (for example implemented in the cloud server) such as a laptop, handheld device or the like. The cloud server in one example implementation can be the AI model Claude 2.0™ is a part of Amazon bedrock. Further, camera on the user-end device can capture video feed of the user to be transmitted to the system 100 in the cloud.

The memory 102 may include any computer-readable medium known in the art including, for example, volatile memory, such as static random access memory (SRAM) and dynamic random access memory (DRAM), and/or non-volatile memory, such as read only memory (ROM), erasable programmable ROM, flash memories, hard disks, optical disks, and magnetic tapes.

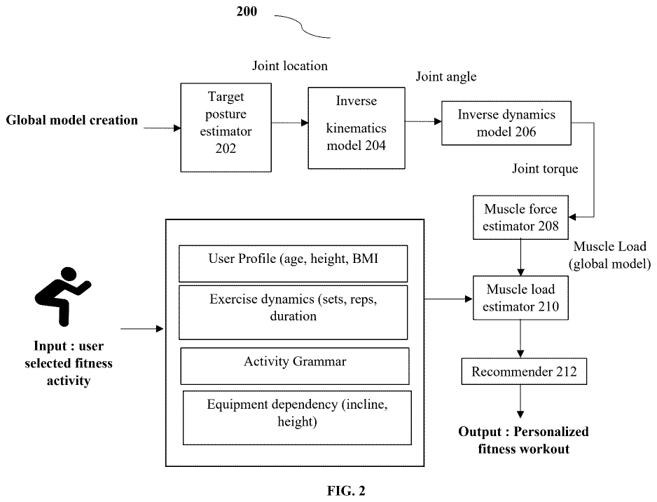

In an embodiment, the memory 102 includes a plurality of modules 110 such as an target posture estimator 202, an inverse kinematics model 204, an inverse dynamics model 206, a muscle force estimator 208 and so on as depicted in FIG. 2. The plurality of modules 110 include programs or coded instructions that supplement applications or functions performed by the system 100 for executing different steps involved in the process of assessing physical risks from geospatial data associated with geographical region being performed by the system 100. The plurality of modules 110, amongst other things, can include routines, programs, objects, components, and data structures, which performs particular tasks or implement particular abstract data types. The plurality of modules 110 may also be used as, signal processor(s), node machine(s), logic circuitries, and/or any other device or component that manipulates signals based on operational instructions. Further, the plurality of modules 110 can be used by hardware, by computer-readable instructions executed by the one or more hardware processors 104, or by a combination thereof. The plurality of modules 110 can include various sub-modules (as shown in FIG. 2).

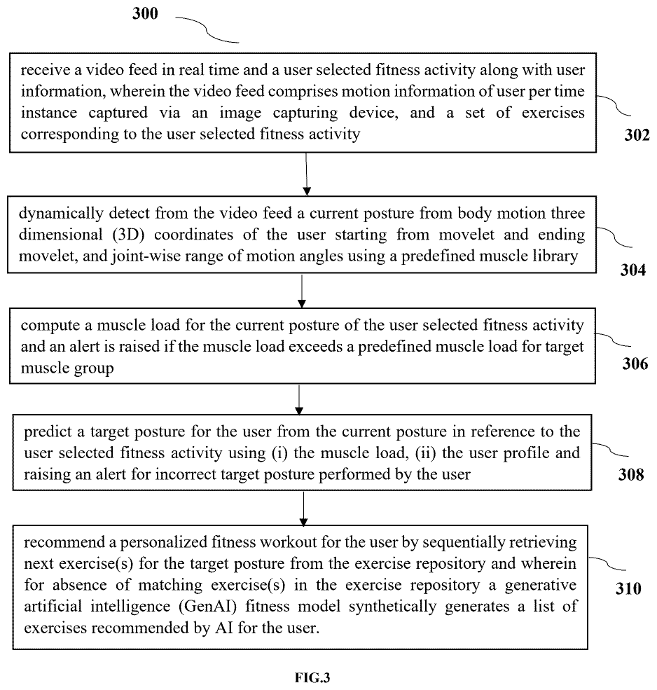

Further, the memory 102 may comprise information pertaining to input(s)/output(s) of each step performed by the processor(s) 104 of the system 100 and methods of the present disclosure. Further, the memory 102 includes a database 108. Although the database 108 is shown internal to the system 100, it will be noted that, in alternate embodiments, the database 108 can also be implemented external to the system 100, and communicatively coupled to the system 100. The data contained within such external database may be periodically updated. For example, new data may be added into the database (not shown in FIG. 1) and/or existing data may be modified and/or non-useful data may be deleted from the database. In one example, the data may be stored in an external system, such as a Lightweight Directory Access Protocol (LDAP) directory and a Relational Database Management System (RDBMS). Functions of the components of the system 100 are now explained with reference to FIG. 2 and steps in flow diagrams in FIG. 3.

FIG. 2 is a functional block diagram illustrating architecture overview of the system 100 for the personalized fitness workout, in accordance with some embodiments of the present disclosure.

Referring to an example where the user aims to perform fitness workout for health benefits with considerable preferences. Here, the user feeds a set of inputs to the system 200 such as user selected fitness activity, video feed and an user profile to recommend artificial intelligence (AI) personalized fitness workout. The user profile is an identifier of the user who performs the fitness activity or exercise(s). User profile is created for a user in the system 100. The user profile captures user information, which is acquired via one or more techniques well known in the art basic health information for example age, gender, BMI entered during registration, date and time the exercise was performed, workout, exercise being performed (squats, lunges, burpees, specific yoga asana, jumping jacks, high knee, mountain climbers, bench press, stationary bike, elliptical, walking, jogging, running, cable etc.), 2D/3D coordinates of body motion points of starting movelet, 2D/3D coordinates of body motion points of ending movelet, joint-wise range of motion angles, hip joint range of motion angle quantified in degrees (.), knee joint range of motion angle quantified in degrees (.), ankle joint range of motion angle quantified in degrees (.), shoulder joint range of motion angle quantified in degrees (.), rom in all three axis, flexion-extension, rotation, abduction-adduction, elbow joint range of motion angle quantified in degrees (.), wrist joint range of motion angle quantified in degrees (.). rom in all three axis, flexion-extension, rotation, abduction-adduction, neck joint range of motion angle quantified in degrees (.), lumbar joint for bending (Flexion-extension), jump height, additional weight (like dumbbell/kettlebell/barbell weightlifting, resistance machines, treadmills, stationary bikes, cable, machine etc.), Impact duration (in seconds), Movement type (flexion) and the like.

For the user selected fitness activity, initially, a target posture estimator 202 integrated into the system 100 (AI cloud) receives the input video feed from the user in real time along with the user selected fitness activity. The fitness activity may be for example exercises (squats, lunges, burpees, specific yoga asana, jumping jacks, high knee, mountain climbers, bench press, stationary bike, elliptical, walking, jogging, etc.).

The target posture estimator 202 detects a current target posture of the user performing activity matching with exercises part of the workout prestored in a repository.

Initially a global model is created having an exercise repository with probable muscle loads. It is understood that global musculoskeletal model is a full body model with 19 degree of freedom in torso and lower extremity, powered by 92 muscle actuators, created in open Sim platform. The biomechanical model is a linked rigid structure consisting of multiple bodies, following human biomechanical constraints. The model is best suited to simulate and analyze lower limb activities.

Further, an inverse kinematics model 204 computes one or more joint angle trajectories from the video feed of the user. Then, an inverse dynamics model 206 computes joint torques from the one or more joint angle trajectories, and a muscle force estimator 208 estimates the muscle force of the user performing an exercise using the joint torques.

Then, a muscle load estimator 210 estimates the muscle load from the muscle force and finally a recommender 212 recommends personalized fitness activity workout for the user.

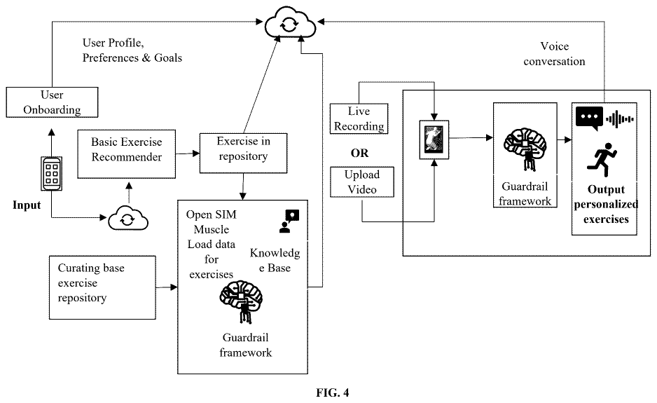

FIG. 3 is a flow diagram illustrating a method to recommend personalized fitness workout using the system of FIG. 2, in accordance with some embodiments of the present disclosure. In an embodiment, the system 100 comprises one or more data storage devices or the memory 102 operatively coupled to the processor(s) 104 and is configured to store instructions for execution of steps of the method 300 by the processor(s) or one or more hardware processors 104. The steps of the method 300 of the present disclosure will now be explained with reference to the components or blocks of the system 100 as depicted in FIG. 1 and FIG. 2, the steps of flow diagram as depicted in FIG. 3 and a use case example of physical risk assessment scenario in FIG. 4. Although process steps, method steps, techniques or the like may be described in a sequential order, such processes, methods, and techniques may be configured to work in alternate orders. In other words, any sequence or order of steps that may be described does not necessarily indicate a requirement that the steps to be performed in that order. The steps of processes described herein may be performed in any order practical. Further, some steps may be performed simultaneously.

Referring to FIG. 3 and the steps of the method 300, at step 302 of the method 300, the one or more hardware processors 104 of the system 100 are configured by the instructions to receive a video feed in real time and an user selected fitness activity along with user information. The video feed comprises motion information of user per time instance captured via an image capturing device, and a set of exercises corresponding to the user selected fitness activity.

Referring to the above example and FIG. 4 where the user installs the application into any user end device for example camera of smart phone, smart TV, laptop, PC, tablet (iPad or android tablet). The activity, the user is interested in performing is received via the user-end device which is transmitted to the system 100 for analysis. For example, the user may select the activity may be squat exercise commonly used in rehabilitation and sport performance settings to strengthen primary lower extremity muscle groups (hip extensors and knee extensors). The prominent muscle groups used in squatting are Quadriceps, Hamstring and Gluteus group of muscles. Along with the user selected fitness activity, input video feed and the user profile are fetched by the system100. An relevant video feed for the selected activity is obtained from repository present in the system 100.

At step 304 of the method 300, the one or more hardware processors 104 is configured to dynamically detect from the video feed a current posture from body motion three dimensional (3D) coordinates of the user starting from movelet and ending movelet, and joint-wise range of motion angles using a predefined muscle library. The predefined muscle library is a library of probable muscle forces created for each activity from the global musculoskeletal model.

Referring to the above example, in the video feed the “squat” as the user selected fitness activity, the target posture estimator 202 detects the current posture body motion three dimensional (3D) coordinates.

For the user interested in performing squat exercise, selection of weight factor varies from age and proficiency level of the user. This is obtained from the global model that is generated prior to system being used for recommendations. The An exercise repository is created for different activity set, the muscle forces in the squat group will be fetched as the starting value.

For person specific applications and tuning, the base muscle forces are fetched from the global database created and adjusted with subject specific anthropometric (height, weight, BMI, age, gender, hip to waist ratio, etc.) and exercise data (like the exercise variant, sets, reps, cycle, pose hold duration, equipment incline (like incline of treadmill while running/walking or bench while doing bench press), body weight/free weight/machine resistance etc.) E.g., plates/disk weight etc.,), free-weights (dumbbells, barbells, kettlebells, medicine balls etc.), resistance in exercise bikes etc. Later using objects identified using computer weights, non-standard weights may also be considered in muscle load calculations.

Then, a weighted matrix is created based on BMI which is alpha α and a hip to waist ratio adjustment as beta β. The weight matrix will be defined combining the age (in years) and exercise proficiency level (fitness) of the user as shown in Table 1,

TABLE 1
Age fitness weight matrix

| | 18-29 | 30-39 | 40-49 | 50-59 | 60+ |
|---|---|---|---|---|---|
| | years | years | years | years | years |
| Pro | W₁₁ | W₁₂ | W₁₃ | W₁₄ | W₁₅ |
| Mid | W₂₁ | W₂₂ | W₂₃ | W₂₄ | W2₅ |
| Novice | W₃₁ | W₃₂ | W₃₃ | W₃₄ | W3₅ |

The values of the weight matrix will be learned from a cohort data set for that particular activity as shown in Table 2,

TABLE 2
BMI (α) Hip to waist ratio (β) weight matrix

| | ≤25 | 30 | ≥30 |
|---|---|---|---|
| 0.85 | αβ₁₁ | αβ₁₂ | αβ₁₃ |
| 0.85-0.89 | αβ₂₁ | αβ₂₂ | αβ₂₃ |
| 0.9-0.95 | αβ₃₁ | αβ₃₂ | αβ₃₃ |

Like the previous weight matrix, this matrix will also be gender specific and values will be learned from cohort data.

Now at step 306 of the method 300, the one or more hardware processors 104 is configured to compute a muscle load for the current posture of the user selected fitness activity, wherein an alert is raised if the muscle load exceeds a predefined muscle load for target muscle group.

Once the current posture is detected from the above step, the muscle load is computed to predict next exercise. However, stamina while performing exercise or activity differs from person to person. Here, the muscle load is computed for the above example “squat”, For the selected fitness activity ‘squat’, and for each fitness activity the inverse kinematics (IK) model 204 computes at every time step, a joint angle trajectory from the 3D motion coordinates of each fitness activity mapped in reference to ground truth of the user selected fitness activity. The joint angles of each fitness activity maps at least one of body joints. The current posture coordinates (p) are function of joint angles (q) and the IK is defined as in Equation 1,

$${P}_{d} - f ( {θ}_{d} ) = 0 \qquad Equation 1$$

θ_{d} is determined using a Newton Raphson method (conventional technique) which is an array of all joints under consideration as in Equation 2,

$${P}_{n + 1} = {P}_{n} {\vert - {( \frac{\partial {F}_{( p )}}{\partial p} )}^{- 1} \vert}_{{P}_{n}} F ( {P}_{n} ) \qquad Equation 2$$

$$\frac{\partial {F}_{( p )}}{\partial p}$$

is an Jacobian matrix which gives the estimated joint angle. For the global model, the estimated coordinates can be matched with experimental marker data. At each time step, generalized coordinate values which positions the model in a pose that “best matches” experimental marker and coordinate values for that time step is selected using least square method. Output of this step is the activity mapped joint angles (knee, hip, ankle, elbow, shoulder, etc.) for any activity over the activity duration.

Then, an Inverse dynamics (ID) model 206 computes the joint torques using the joint angle trajectory obtained from the IK model and ground reaction force is measured from ground reaction force sensor. Joint torques are calculated using Equation 3,

$$τ = M ( q ) \ddot{q} + C ( ( q ) \dot{q} ) + G ( q ) + F \qquad Equation 3$$

where, \ddot{q}, \dot{q}, and q are the acceleration, velocity and position due to joint torque τ, M is the mass matrix, C and G are the Coriolis and gravity component and F is any external force applied to the model. The resultant output is an array of desired joint torques (hip, knee, ankle, etc.).

Further, a muscle force estimator 208 estimates a muscle force using the joint torques, a moment arm [R(q)], an activation value a and a normalized length (l) of the muscle unit. The muscle forces are generated in the model are based on the ‘Thelen muscle’ actuator, where the muscle force or torque component (τₘ) is calculated using the in Equation 4,

$${τ}_{m} = [ R ( q ) f ( a , l , l ) \qquad Equation 4$$

[R(q)] is the moment arm, a is the activation value and l is the normalized length of the muscle unit. Muscle forces over one gait cycle were estimated using the static optimization algorithm (SO). The muscle force is estimated by minimizing the sum of squared muscle activations generated using the Thelen muscle model that is required to drive experimentally captured kinematics and ground reaction force at each time instance.

Finally, the muscle load estimator 210 computes the muscle load using a muscle force, a number of muscles (n) in the model, an activation level (aₘ) of muscle m at a discrete time step, an maximum isometric force

$${F}_{m}^{0} ,$$

a length (l), a shortening velocity (vₘ), a moment arm j about the jth joint axis, and a generalized force (τⱼ) acting about the jth joint axis.

The Static Optimization Tool uses the known motion of the model (from IK) to solve the equations of motion for the unknown generalized forces (τₘ) subject to constrained by force-length-velocity properties of muscle force, that achieves minimization of an objective function (muscle activation profile). The corrected muscle force is computed as in Equation 5,

$${\sum }_{m = 1}^{n} [ {a}_{m} f ( {F}_{m}^{0} , {l}_{m} , {v}_{m} ) ] {r}_{m , j} = {τ}_{j} \qquad Equation 5$$

where n is the number of muscles in the model, aₘ is the activation level of muscle m at discrete time step,

$${F}_{m}^{0}$$

is the maximum isometric force, lₘ is the length, vₘ is the shortening velocity, r_{m,j} is the moment arm about the jth joint axis, τⱼ is the generalized force acting about the jth joint axis. Muscle activation generated in the model can be validated using the muscle activity recordings using EMG sensors in lab. Output of this step is the muscle force for all 92 muscle actuators for that particular activity. These steps concludes computation of the muscle force from the global MSK model. For different activity, such muscle force will be computed to form a baseline database. Next, for specific activity and persona, muscle loads are computed taking the initial muscle force from the database created by the global model. The muscle force for any activity will be a function of the user inputs dynamic activity parameters and machine setting, if applicable like treadmill inclination. The steps are provided next considering a specific use case of dead weight squat exercise.

At step 308 of the method 300, the one or more hardware processors 104 is configured to predict a target posture for the user from the current posture in reference to the user selected fitness activity using (i) the muscle load, (ii) the user profile and an alert is raised for incorrect target posture performed by the user, wherein the target posture is obtained from a exercise repository based on a matching criteria from pretrained biomechanical models, a correlation between the current posture of the user and the user selected fitness activity.

Further, the target posture for the user is predicted by obtaining the user profile and the user selected fitness activity as categorical ranked list. Here, a current joint posture range of joint motion angles is obtained and increase in muscle load deviated from the target muscle group.

The matching criteria is a closest match of the current posture of the user in reference to the target posture obtained from a pretrained biomechanical models and the user profile.

Initially, a first rank list M₁ is created by extracting matching next target posture to be performed by the user from the exercise repository based on TF/IDF followed by cosine similarity. Then, a second rank list M₂ is created by extracting matching next target posture from an user testimonial, Finally, a final match M is predicted using a complimentary filter based on the first rank list and the second rank list as described in Equation 6,

$$M = α * {M}_{1} + ( 1 - α ) * {M}_{2} \qquad Equation 6$$

wherein, the predictions are ranked according to increasing value of M and initially alpha is set to 1 for low number of user and the user testimonials adds a to a target value of 0.8 using a epsilon greedy algorithm.

Muscle load is computed for a person doing the squat exercise for these muscle groups. Muscle load estimation takes dynamic exercise information such as no of sets (n), no of repetition (reps)/set, duration of 1 set, resting time between set. Muscle load for ‘n’ number of sets for each muscle group (quad, ham and gluteus) under consideration will be computed as in Equation 7,

$${M}_{L} = \frac{1}{BW} Factor {\sum }_{i = 1}^{n} [ {W}_{i , j αβ} {\int }_{0}^{{t}_{r}} τ {Mdt}_{r} ] + αβ {τ}_{M} ( {e}^{- RT} ) \qquad Equation 7$$

Where, BW is the subject body weight, W_{i,jαβ} will be taken from appropriate entry in Table 1, ab is a value from Table 2, τM is the Muscle force computed from the Equation 5, derived from the global model, dtᵣ is the total repetitions (reps) time. Factor is the muscle and exercise specific parameter (to be discussed next). The second function is described in Equation 7 which is the adjustment factor inherent to muscle recovery pattern. RT is the rest time between sets, which is between 2 to 5 mints for squat exercise. Muscle load for one set is subjected to a constraint of max 85% of repetition max (RM) which is the maximum dynamic strength in Kg.

Finally at step 310, the one or more hardware processors 104 is configured to recommend a personalized fitness workout for the user by sequentially retrieving next exercise(s) for the target posture from the exercise repository, and wherein for absence of matching exercise(s) in the exercise repository a generative artificial intelligence (GenAI) fitness model synthetically generates a list of exercises recommended by AI for the user. Once the target posture is predicted from the above step the personalized fitness workout is created based on the recommendations provided from the exercise repository.

In another embodiment, if no matching exercise(s) are identified in the exercise repository, the generative artificial intelligence (GenAI) fitness model synthetically generates a list of exercises when no next best exercise(s) is identified in the exercise repository by performing the following steps, the current posture Pₛ and the target posture Pₜ to be reached is obtained. Further, a reward function is assigned to a path to reach the target posture Pₜ for the action, wherein the reward function includes two parts and a Q-learning state-space s is constructed using the model m. The reward function is designed with two parts comprising a first part includes a minimum number of movements, and minimum joint movements e^{threshold-number of movements} and a second part includes a maximum movement of target muscles e^{f(muscle load)}. The reward function is computed using the Equation 8,

$$Reward function = ( {e}^{threshold - number of movements} ) + {e}^{f ( muscle load )} \qquad Equation 8$$

Where, f(load muscle)=load on muscle, if load≤max; 0 otherwise. It is noted based on the user's profile max muscle load is represented with +/−5% for the target muscle group is known, and the above state and action pair is used to create a path from Pₛ to Pₜ to predict the next target posture.

Referring to FIG. 4, experimental results factors adjustment for muscle groups of “squat” exercise can be highly variable in its execution. The user may perform same dead weight squat but with varying depth, stance width, trunk inclination, etc. For each such variation, load distribution in the muscle group varies. For example, a positive trunk inclination results in higher muscle load in hip muscles and back muscles. Hence, defined by the pose information (p(x, y, z)) and grammar set for each exercise (squat for this particular example), some load adjustment factors will be defined. Factors includes Trunk inclination, Tibia inclination, squat depth and stance width.

Trunk inclination are the moments at the hip and knee during squatting are highly influenced by the orientation of the trunk, which in turn affects the center of mass of the body. Moving the trunk from a more upright position to more forward position (forward bending) (qt r) shifts the resultant ground reaction force vector anteriorly, resulting in an increase in the hip flexion moment while simultaneously decreasing the knee flexion moment. Factor adjustment for GLUTEUS and HAMSTRING muscle group as in Equation 9,

$${Factor}_{tr} = {θ}_{tr} * 5 \% {M}_{F} \qquad Equation 9$$

for each degree inclination from upright posture, increase eq.6 by a factor defined by Equation 7 for GLUTEUS and decrease Equation 9 by a factor defined by Equation 7 for HAMSTRING and in Equation 9 remains unchanged for Quadriceps for trunk inclination deviation.

Tibia Inclination: Moving the tibia from a more upright position to more forward position shifts the knee joint center further away from the resultant ground reaction force vector, thereby increasing the knee flexion moment. Factor adjustment for only Hamstring muscle group as in Equation 10,

$${Factor}_{tib} = {θ}_{tr} * 3 \% {M}_{F} \qquad Equation 10$$

for each degree inclination from upright posture, increase Equation 7 by a factor defined by Equation 10 for HAMSTRING.

Depth of Squat: Squat depth can be defined as partial or shallow (0-90 degree knee flexion), medium (90-110 degree knee flexion or thigh parallel to floor), or full or deep (110-135 degree knee flexion). The knee flexion moment tends to steadily increase from an upright position to maximum knee flexion during squatting. Similarly, the hip flexion moment also increases with squat dept.

For Gluteus muscle group, shallow squat Muscle force as mentioned below in Equation 11,

$${ML}_{{GUT}_{SH}} = \frac{1}{BW} {\sum }_{i = 1}^{n} [ {W}_{i , j αβ} {\int }_{0}^{{t}_{r}} τ {Mdt}_{r} ] + αβ {τ}_{M} ( {e}^{- RT} ) \qquad Equation 11$$

Medium depth is mentioned in Equation 12,

$${ML}_{{GUT}_{ME}} = \frac{1}{BW} 0.3 {\sum }_{i = 1}^{n} [ {W}_{i , j αβ} {\int }_{0}^{{t}_{r}} τ {Mdt}_{r} ] + αβ {τ}_{M} ( {e}^{- RT} ) \qquad Equation 12$$

Deep depth is mentioned in Equation 13,

$${ML}_{{GUT}_{DE}} = \frac{1}{BW} 0.5 {\sum }_{i = 1}^{n} [ {W}_{i , j αβ} {\int }_{0}^{{t}_{r}} τ {Mdt}_{r} ] + αβ {τ}_{M} ( {e}^{- RT} ) \qquad Equation 13$$

Similarly, for quadricep group, adjustment are as described for shallow depth in Equation 14,

$${ML}_{{QUAD}_{SH}} = \frac{1}{BW} {\sum }_{i = 1}^{n} [ {W}_{i , j αβ} {\int }_{0}^{{t}_{r}} τ {Mdt}_{r} ] + αβ {τ}_{M} ( {e}^{- RT} ) \qquad Equation 14$$

Medium depth is mentioned in Equation 15,

$${ML}_{{QUAD}_{ME}} = \frac{1}{BW} 0.15 {\sum }_{i = 1}^{n} [ {W}_{i , j αβ} {\int }_{0}^{{t}_{r}} τ {Mdt}_{r} ] + αβ {τ}_{M} ( {e}^{- RT} ) \qquad Equation 15$$

Deep depth is mentioned in Equation 16,

$${ML}_{{QUAD}_{DE}} = \frac{1}{BW} 0.3 {\sum }_{i = 1}^{n} [ {W}_{i , j αβ} {\int }_{0}^{{t}_{r}} τ {Mdt}_{r} ] + αβ {τ}_{M} ( {e}^{- RT} ) \qquad Equation 16$$

No adjustment for Hamstring, Equation 7 will be computed irrespective of depth variation. Stance Width Stance width can be defined as narrow (75% to 100% shoulder width), medium (100% to 150% shoulder width), or wide 150% to 200% shoulder width.

From a muscle recruitment standpoint, a medium or wide stance squat has been reported to result in higher gluteus maximus activity without much change in hamstring or quad group. Factor adjustment for Gluteus muscle for narrow is null, will follow Equation 7. Muscle load in Gluteus for medium width as in Equation 17

$${ML}_{{QUAD}_{ME}} = \frac{1}{BW} 0.2 {\sum }_{i = 1}^{n} [ {W}_{i , j αβ} {\int }_{0}^{{t}_{r}} τ {Mdt}_{r} ] + αβ {τ}_{M} ( {e}^{- RT} ) \qquad Equation 17$$

Muscle load in Gluteus for wide width as in Equation 18,

$${ML}_{{QUAD}_{W 1}} = \frac{1}{BW} 0.4 {\sum }_{i = 1}^{n} [ {W}_{i , j αβ} {\int }_{0}^{{t}_{r}} τ {Mdt}_{r} ] + αβ {τ}_{M} ( {e}^{- RT} ) \qquad Equation 18$$

These adjustments concludes grammar related muscle load correction for squat activity. It is to be noted that the adjustment factors will vary based on the activity type but the base Equation 7 will remain same for most lower limb activity.

The written description describes the subject matter herein to enable any person skilled in the art to make and use the embodiments. The scope of the subject matter embodiments is defined by the claims and may include other modifications that occur to those skilled in the art. Such other modifications are intended to be within the scope of the claims if they have similar elements that do not differ from the literal language of the claims or if they include equivalent elements with insubstantial differences from the literal language of the claims.

The embodiments of present disclosure herein addresses unresolved problem of personalized fitness workout. The embodiment, thus provides low-latency personalized fitness recommendation using muscle load method and system. Moreover, the embodiments herein further provides to estimate posture and raise warnings for posture as applicable, 2. Perform muscle load computation and provide recommendations/warnings as appropriate, 3. Derive max joint-wise range of motion angles and muscular strength to profile the user, 4. Predict the next best exercise(s) for the user based on their profile, and 5. Synthetically generate the next best exercise(s) for the user using GenAI. The method provides base exercise repository using generative AI which builds capability to arrive at muscle loading and tacit knowledge for a given personalized fitness workout. For each workout, the base AI model is trained with data from motion labs for an individual with multiple heights, weights (and hence BMI), age, gender, hip to waist ratio (body type). The generative AI model can be implemented in Claude 2.0 is a part of Amazon bedrock or another suitable LLM, these base values can be synthesized for any individuals across varied BMI's, weight, and height, age, gender, hip to waist ratio. In addition to muscle loading, the model is trained with expert best practices, do's and don'ts, conditions and suitable workout which forms the tacit knowledge, this provides personalized messages to an individual who is performing a workout.

It is to be understood that the scope of the protection is extended to such a program and in addition to a computer-readable means having a message therein; such computer-readable storage means contain program-code means for implementation of one or more steps of the method, when the program runs on a server or mobile device or any suitable programmable device. The hardware device can be any kind of device which can be programmed including e.g., any kind of computer like a server or a personal computer, or the like, or any combination thereof. The device may also include means which could be e.g., hardware means like e.g., an application-specific integrated circuit (ASIC), a field-programmable gate array (FPGA), or a combination of hardware and software means, e.g., an ASIC and an FPGA, or at least one microprocessor and at least one memory with software processing components located therein. Thus, the means can include both hardware means and software means. The method embodiments described herein could be implemented in hardware and software. The device may also include software means. Alternatively, the embodiments may be implemented on different hardware devices, e.g., using a plurality of CPUs.

The embodiments herein can comprise hardware and software elements. The embodiments that are implemented in software include but are not limited to, firmware, resident software, microcode, etc. The functions performed by various components described herein may be implemented in other components or combinations of other components. For the purposes of this description, a computer-usable or computer readable medium can be any apparatus that can comprise, store, communicate, propagate, or transport the program for use by or in connection with the instruction execution system, apparatus, or device.

The illustrated steps are set out to explain the exemplary embodiments shown, and it should be anticipated that ongoing technological development will change the manner in which particular functions are performed. These examples are presented herein for purposes of illustration, and not limitation. Further, the boundaries of the functional building blocks have been arbitrarily defined herein for the convenience of the description. Alternative boundaries can be defined so long as the specified functions and relationships thereof are appropriately performed. Alternatives (including equivalents, extensions, variations, deviations, etc., of those described herein) will be apparent to persons skilled in the relevant art(s) based on the teachings contained herein. Such alternatives fall within the scope of the disclosed embodiments. Also, the words “comprising,” “having,” “containing,” and “including,” and other similar forms are intended to be equivalent in meaning and be open ended in that an item or items following any one of these words is not meant to be an exhaustive listing of such item or items, or meant to be limited to only the listed item or items. It must also be noted that as used herein and in the appended claims, the singular forms “a,” “an,” and “the” include plural references unless the context clearly dictates otherwise.

Furthermore, one or more computer-readable storage media may be utilized in implementing embodiments consistent with the present disclosure. A computer-readable storage medium refers to any type of physical memory on which information or data readable by a processor may be stored. Thus, a computer-readable storage medium may store instructions for execution by one or more processors, including instructions for causing the processor(s) to perform steps or stages consistent with the embodiments described herein. The term “computer-readable medium” should be understood to include tangible items and exclude carrier waves and transient signals, i.e., be non-transitory. Examples include random access memory (RAM), read-only memory (ROM), volatile memory, nonvolatile memory, hard drives, CD ROMs, DVDs, flash drives, disks, and any other known physical storage media.

It is intended that the disclosure and examples be considered as exemplary only, with a true scope of disclosed embodiments being indicated by the following claims.

What is claimed is:

1. A processor implemented method for personalized fitness recommendation workout, the method comprising:
receiving via one or more hardware processors, a video feed in real time and an user selected fitness activity along with user information, wherein the video feed comprises motion information of user per time instance captured via an image capturing device, and a set of exercises corresponding to the user selected fitness activity;
dynamically via the one or more hardware processors, detecting from the video feed, a current posture from body motion three dimensional (3D) coordinates of the user starting from movelet and ending movelet, and joint-wise range of motion angles using a predefined muscle library;
computing via the one or more hardware processors, a muscle load for the current posture of the user selected fitness activity, wherein an alert is raised if the muscle load exceeds a predefined muscle load for target muscle group;
predicting via the one or more hardware processors, a target posture for the user from the current posture in reference to the user selected fitness activity using (i) the muscle load, (ii) the user profile and raising an alert for incorrect target posture performed by the user, wherein the target posture is obtained from a exercise repository based on a matching criteria from pretrained biomechanical models, a correlation between the current posture of the user and the user selected fitness activity; and
recommending via the one or more hardware processors, a personalized fitness workout for the user by sequentially retrieving next matching exercise(s) for the target posture from the exercise repository, and wherein for absence of matching exercise(s) in the exercise repository a generative artificial intelligence (GenAI) fitness model synthetically generates a list of exercises recommended by AI for the user.

2. The processor implemented method of claim 1, wherein the matching criteria is a closest match of the current posture of the user in reference to the target posture obtained from a pretrained biomechanical models and the user profile.

3. The processor implemented method of claim 1, wherein the muscle load is computed by,
computing using an inverse kinematics (IK) model at every time step joint angle trajectory from the 3D motion coordinates of each fitness activity mapped in reference to ground truth of the user selected fitness activity, wherein the joint angles of each fitness activity maps at least one of body joints;
computing using an inverse dynamics (ID) model the joint torques using the joint angle trajectory obtained from the IK model and measuring ground reaction force from ground reaction force sensor;
computing a muscle force using the joint torques, a moment arm [R(q)], an activation value a and a normalized length (l) of the muscle unit; and
computing the muscle load using a muscle force, a number of muscles (n) in the model, an activation level (aₘ) of muscle m at a discrete time step, an maximum isometric force
$${F}_{m}^{0} ,$$
a length (l), a shortening velocity (vₘ), a moment arm j about the jth joint axis, and a generalized force (τⱼ) acting about the jth joint axis.

4. The processor implemented method of claim 1, wherein the target posture is predicted for the user selected fitness activity by performing the steps of:
obtaining the user profile and the user selected fitness activity as categorical ranked list;
obtaining a current joint posture range of joint motion angles and increase in muscle load deviated from the target muscle group;
creating a first rank list M₁ by extracting matching next target posture to be performed by the user from the exercise repository based on TF/IDF followed by cosine similarity;
creating a second rank list M₂ by extracting matching next target posture from an user testimonial; and
predicting a final match M, using a complimentary filter based on the first rank list and the second rank list M=α*M₁+(1−α)*M₂, wherein the predictions are ranked according to increasing value of M and setting the α value to one for low number of user otherwise for the user testimonials α is added with a target value to 0.8 using a epsilon greedy algorithm.

5. The processor implemented method of claim 1, wherein the generative artificial intelligence (GenAI) fitness model synthetically generates a list of exercises in absence of next best exercise(s) by,
obtaining the current posture Pₛ and the target posture Pₜ to be reached;
assigning a reward function to a path to reach the target posture Pₜ for the action, wherein the reward function includes two parts; and
constructing a Q-learning state-space s using the model m.

6. The processor implemented method of claim 5, wherein the reward function is a sum of two parts comprising a first part includes a minimum number of movements and minimum joint movements (e^{threshold-number of movements}) and a second part includes a maximum movement of target muscles e^{f(muscle load)}, where f(load muscle)=load on muscle, if load≤max; 0 otherwise.

7. A system, for personalized fitness recommendation workout comprising:
a memory storing instructions;
one or more communication interfaces; and
one or more hardware processors coupled to the memory via the one or more communication interfaces, wherein the one or more hardware processors are configured by the instructions to:
receive a video feed in real time and an user selected fitness activity along with user information, wherein the video feed comprises motion information of user per time instance captured via an image capturing device, and a set of exercises corresponding to the user selected fitness activity;
dynamically detect from the video feed, a current posture from body motion three dimensional (3D) coordinates of the user starting from movelet and ending movelet, and joint-wise range of motion angles using a predefined muscle library;
compute a muscle load for the current posture of the user selected fitness activity, wherein an alert is raised if the muscle load exceeds a predefined muscle load for target muscle group;
predict a target posture for the user from the current posture in reference to the user selected fitness activity using (i) the muscle load, (ii) the user profile and raising an alert for incorrect target posture performed by the user, wherein the target posture is obtained from a exercise repository based on a matching criteria from pretrained biomechanical models, a correlation between the current posture of the user and the user selected fitness activity; and
recommend a personalized fitness workout for the user by sequentially retrieving next matching exercise(s) for the target posture from the exercise repository, and wherein for absence of matching exercise(s) in the exercise repository a generative artificial intelligence (GenAI) fitness model synthetically generates a list of exercises recommended by AI for the user.

8. The system of claim 7, wherein the matching criteria is a closest match of the current posture of the user in reference to the target posture obtained from a pretrained biomechanical models and the user profile.

9. The system of claim 7, wherein the muscle load is computed by,
compute using an inverse kinematics (IK) model at every time step joint angle trajectory from the 3D motion coordinates of each fitness activity mapped in reference to ground truth of the user selected fitness activity, wherein the joint angles of each fitness activity maps at least one of body joints;
computing using an inverse dynamics (ID) model the joint torques using the joint angle trajectory obtained from the IK model and measuring ground reaction force from ground reaction force sensor;
computing a muscle force using the joint torques, a moment arm [R(q)], an activation value a and a normalized length (l) of the muscle unit; and
computing the muscle load using a muscle force, a number of muscles (n) in the model, an activation level (aₘ) of muscle m at a discrete time step, an maximum isometric force
$${F}_{m}^{0} ,$$
a length (l), a shortening velocity (vₘ), a moment arm j about the jth joint axis, and a generalized force (τⱼ) acting about the jth joint axis.

10. The system of claim 7, wherein the target posture is predicted for the user selected fitness activity by performing the steps of:
obtaining the user profile and the user selected fitness activity as categorical ranked list;
obtaining a current joint posture range of joint motion angles and increase in muscle load deviated from the target muscle group;
creating a first rank list M₁ by extracting matching next target posture to be performed by the user from the exercise repository based on TF/IDF followed by cosine similarity;
creating a second rank list M₂ by extracting matching next target posture from an user testimonial; and
predicting a final match M, using a complimentary filter based on the first rank list and the second rank list M=α*M₁+(1−α)*M₂, wherein the predictions are ranked according to increasing value of M and setting the α value to one for low number of user otherwise for the user testimonials α is added with a target value to 0.8 using a epsilon greedy algorithm.

11. The system of claim 7, wherein the generative artificial intelligence (GenAI) fitness model synthetically generates a list of exercises in absence of next best exercise(s) by,
obtaining the current posture Pₛ and the target posture Pₜ to be reached;
assigning a reward function to a path to reach the target posture Pₜ for the action, wherein the reward function includes two parts;
and
constructing a Q-learning state-space s using the model m.

12. The system of claim 11, wherein the reward function is a sum of two parts comprising a first part includes a minimum number of movements and minimum joint movements (e^{threshold-number of movements}) and a second part includes a maximum movement of target muscles e^{f(muscle load)}, where f(load muscle)=load on muscle, if load≤max; 0 otherwise.

13. One or more non-transitory machine-readable information storage mediums comprising one or more instructions which when executed by one or more hardware processors cause:
receiving, a video feed in real time and an user selected fitness activity along with user information, wherein the video feed comprises motion information of user per time instance captured via an image capturing device, and a set of exercises corresponding to the user selected fitness activity;
dynamically, detecting from the video feed, a current posture from body motion three dimensional (3D) coordinates of the user starting from movelet and ending movelet, and joint-wise range of motion angles using a predefined muscle library;
computing, a muscle load for the current posture of the user selected fitness activity, wherein an alert is raised if the muscle load exceeds a predefined muscle load for target muscle group;
predicting, a target posture for the user from the current posture in reference to the user selected fitness activity using (i) the muscle load, (ii) the user profile and raising an alert for incorrect target posture performed by the user, wherein the target posture is obtained from a exercise repository based on a matching criteria from pretrained biomechanical models, a correlation between the current posture of the user and the user selected fitness activity; and
recommending, a personalized fitness workout for the user by sequentially retrieving next matching exercise(s) for the target posture from the exercise repository, and wherein for absence of matching exercise(s) in the exercise repository a generative artificial intelligence (GenAI) fitness model synthetically generates a list of exercises recommended by AI for the user.

14. The one or more non-transitory machine readable information storage mediums of claim 13, wherein the matching criteria is a closest match of the current posture of the user in reference to the target posture obtained from a pretrained biomechanical models and the user profile.

15. The one or more non-transitory machine readable information storage mediums of claim 13, wherein the muscle load is computed by,
computing using an inverse kinematics (IK) model at every time step joint angle trajectory from the 3D motion coordinates of each fitness activity mapped in reference to ground truth of the user selected fitness activity, wherein the joint angles of each fitness activity maps at least one of body joints;
computing using an inverse dynamics (ID) model the joint torques using the joint angle trajectory obtained from the IK model and measuring ground reaction force from ground reaction force sensor;
computing a muscle force using the joint torques, a moment arm [R(q)], an activation value a and a normalized length (l) of the muscle unit; and
computing the muscle load using a muscle force, a number of muscles (n) in the model, an activation level (aₘ) of muscle m at a discrete time step, an maximum isometric force
$${F}_{m}^{0} ,$$
a length (l), a shortening velocity (vₘ), a moment arm j about the jth joint axis, and a generalized force (τⱼ) acting about the jth joint axis.

16. The one or more non-transitory machine readable information storage mediums of claim 13, wherein the target posture is predicted for the user selected fitness activity by performing the steps of:
obtaining the user profile and the user selected fitness activity as categorical ranked list;
obtaining a current joint posture range of joint motion angles and increase in muscle load deviated from the target muscle group;
creating a first rank list M₁ by extracting matching next target posture to be performed by the user from the exercise repository based on TF/IDF followed by cosine similarity;
creating a second rank list M₂ by extracting matching next target posture from an user testimonial; and
predicting a final match M, using a complimentary filter based on the first rank list and the second rank list M=α*M₁+(1−α)*M₂, wherein the predictions are ranked according to increasing value of M and setting the α value to one for low number of user otherwise for the user testimonials α is added with a target value to 0.8 using a epsilon greedy algorithm.

17. The one or more non-transitory machine readable information storage mediums of claim 13, wherein the generative artificial intelligence (GenAI) fitness model synthetically generates a list of exercises in absence of next best exercise(s) by,
obtaining the current posture Pₛ and the target posture Pₜ to be reached;
assigning a reward function to a path to reach the target posture Pₜ for the action, wherein the reward function includes two parts; and
constructing a Q-learning state-space s using the model m.

18. The one or more non-transitory machine readable information storage mediums of claim 17, wherein the reward function is a sum of two parts comprising a first part includes a minimum number of movements and minimum joint movements (e^{threshold-number of movements}) and a second part includes a maximum movement of target muscles e^{f(muscle load)}, where f(load muscle)=load on muscle, if load≤max; 0 otherwise.
